(19) United States
(12) Patent Application Publication
(10) Pub. No.: US 2022/0175676 A1
(43) Pub. Date: Jun. 9, 2022

(54) MULTIFUNCTIONAL POLYMER-NANOPARTICLE COMPOSITE FOR FIRST AID AND WOUND CARE APPLICATIONS

(71) Applicant: NSC – NANO SONO COOPERATION LTD, Yokneam Illit (IL)

(72) Inventors: Rajashekharayya A. Sanguramath, Yokneam Illit (IL); Ariel Antonio Franco, Yokneam Illit (IL)

(73) Assignee: NSC – NANO SONO COOPERATION LTD, Yokneam Illit (IL)

(21) Appl. No.: 17/540,282

(22) Filed: Dec. 2, 2021

Related U.S. Application Data
(60) Provisional application No. 63/120,788, filed on Dec. 3, 2020.

(51) Int. Cl. A61K 9/14; A61K 9/00; A61F 13/00; A61K 45/06

(52) U.S. Cl. CPC A61K 9/143; A61K 9/146; B82Y 5/00; A61F 13/00063; A61K 45/06; A61K 9/0014

(57) ABSTRACT

Described herein is a multifunctional polymer-nanoparticle composition for use in wound care applications. Methods of manufacturing the described compositions are also disclosed herein.

### CROSS-REFERENCE TO RELATED APPLICATIONS

Benefit is claimed of U.S. Provisional Patent Application No. 63/120,788, filed Dec. 3, 2020, the contents of which are incorporated by reference herein in their entirety.

### FIELD

The present disclosure relates to a multifunctional polymer-nanoparticle composition for use in wound care applications. Methods of manufacturing the described compositions are also disclosed herein.

### BACKGROUND

Wound care is a rapidly evolving field. Although humans have treated small and large wounds for millennia, the materials used for first aid and long-term care as dressings to cover and facilitate wound healing have seen many recent advances (Dhivya et al., BioMedicine 5:24-28, 2015).

Metal oxide nanomaterials and their composites are one example of materials proposed to provide antimicrobial and drug depot functionalities (Matter et al., Pharmaceutics 12 (780), 2020). Other materials, including certain biopolymers are also in development for use in or as wound dressings (Dhivya et al.).

Layered nanoparticle composites have been developed for a variety of applications (see for examples U.S. Pat. Nos. 10,278,927 and 8,685,538). However, none of the layered nanoparticles proposed to date have united multiple functionalities that together would provide a significant advancement in first and long-term wound care. Thus, a continuing need exists for development of materials to provide more effective wound care.

### SUMMARY

Described herein are compositions for use in treating a wound that include an antimicrobial metal oxide nanoparticle; a first polymer layer coating the antibacterial metal oxide nanoparticle; an external polymer layer coating the polymer-coated nanoparticle; and optionally at least one additional polymer layer between the first polymer layer and the external polymer layer, wherein the first polymer layer is optionally a hemostasis-promoting polymer and/or has been loaded with a pharmaceutical agent; and wherein the external polymer layer and the optional at least one additional polymer layer is a hemostasis-promoting polymer, mucoadhesive polymer, and/or has been loaded with a pharmaceutical agent that is the same or different from the pharmaceutical agent of the first polymer layer. Methods of treatment of a wound, such as in first-aid or long-term wound care, by contacting a wound with the described compositions, are also provided herein.

The foregoing and other objects, features, and advantages will become more apparent from the following detailed description, which proceeds with reference to the accompanying figures.

### BRIEF DESCRIPTION OF THE DRAWINGS

FIG. 1 is a graph showing the ζ-potential shift upon addition of a modified chitosan layer to a calcium alginate-coated CuO₍₁₋ₓ₎ZnOₓ antibacterial nanocomposite. The alginate-coated nanoparticle peaks at −16 mV. The nanoparticle coated with both calcium alginate and modified chitosan layers peaks at +16 mV.

FIG. 2 is a graph showing thermogravimetric analysis for measuring mass percentage of different polymers in the synthesized polymer-CuO₍₁₋ₓ₎ZnOₓ antibacterial nanocomposites. The top curve is the calcium alginate-coated nanoparticle composite. The lower curve is the nanoparticle coated with both calcium alginate and modified chitosan layers.

FIG. 3 is a graph showing alternation of ζ-potential as a function of negatively charged alginate and positively charged chitosan polymer deposition on CuO₍₁₋ₓ₎ZnOₓ antibacterial nanocomposite.

FIG. 4 is a graph showing TGA curves for multi-layers of polymers coated CuO₍₁₋ₓ₎ZnOₓ antibacterial nanocomposite. The curves representing layers 1, 2, 3, 4, and 5 are shown from the top to the bottom in order.

FIG. 5 is a graph showing the Ibuprofen release profile in pH 7.4 phosphate buffer from 2 g/L concentration of chitosan-alginate coated CuO₍₁₋ₓ₎ZnOₓ antibacterial nanocomposite. Chitosan is the second layer, and the Ibuprofen was added to the chitosan layer.

FIG. 6 is a graph showing the Ibuprofen release profile in pH 7.4 phosphate buffer from 10 g/L concentration of chitosan-alginate coated CuO₍₁₋ₓ₎ZnOₓ antibacterial nanocomposite. Chitosan is the second layer, and the Ibuprofen was added to the chitosan layer.

FIG. 7 is a graph showing the Tetracaine release profile in pH 7.4 phosphate buffer from 10 g/L concentration of alginate-chitosan coated CuO₍₁₋ₓ₎ZnOₓ antibacterial nanocomposite. Alginate is the second layer, and the tetracaine was added to the alginate layer.

FIG. 8 are high resolution transmission electron microscope (HRTEM) images showing tetracaine loaded Alginate-Chitosan-Alginate-Chitosan coated CuO₍₁₋ₓ₎ZnOₓ. Tetracaine was loaded to alginate layers. Images at 50 nm (left panel) and 10 nm (right panels) scale are shown.

FIG. 9 is a graph showing the Ibuprofen release profile in pH 7.4 phosphate buffer from 10 g/L concentration of chitosan-alginate-chitosan-alginate coated CuO₍₁₋ₓ₎ZnOₓ antibacterial nanocomposite. Ibuprofen was added to the chitosan layers.

FIG. 10 are photographs showing Proof of concept for coating multi-functional nanocomposite on a first aid wound dressing gauze. Left, first aid gauze before coating with the multi-functional nanocomposite. Right, first aid gauze after coating with the multi-functional nanocomposite based on tetracaine loaded chitosan-alginate-CuO₍₁₋ₓ₎ZnOₓ.

FIG. 11 shows scanning electron micrographs (SEM) showing first aid medical gauze without (top) and with (bottom) multi-functional nanocomposite.

### DETAILED DESCRIPTION

### I. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all molecular weight or molecular mass values are approximate, and are provided for illustrative description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.,” is synonymous with the term “for example.”

In case of conflict, the present specification, including explanations of terms, will control. In addition, all the materials, methods, and examples are illustrative and not intended to be limiting.

Administration: The introduction of a composition into or onto a subject by a chosen route. For example, the described polymer-nanoparticle composite compounds can be administered locally at a wound site by any method known to the art of contacting a surface with a compound.

Antimicrobial agent: A compound that inhibits, prevents, or eradicates the growth, replication, spread or activity of a microorganism. In a particular embodiment, an antimicrobial agent is a metal oxide nanoparticle component of the described polymer-nanoparticle composite compounds. When used generally, an antimicrobial agent can inhibit, prevent, or eradicate the growth and spread of living microbes such as bacteria and fungi. Similarly, an antimicrobial agent can also inhibit the viability of a viral particle to infect and successfully replicate within a host, thereby eradicating its presence from the host. A microbe may be inhibited when its presence or activity is decreased by at least 10%, at least 20%, at least 30%, at least 50%, at least 80%, at least 100% or at least 250% or more as compared to a microbe that has not been contacted with the compound.

Contacting: Placement in direct physical association; including contact of a surface by a composition both in solid and liquid forms. Contacting can occur in vivo by administering to a subject.

Composite: A material composed of two or more constituent parts, which are generally structurally and physically distinct.

Effective amount of a compound: A quantity of compound sufficient to achieve a desired effect. In a therapeutic context, a therapeutically effective amount of a compound is that amount to achieve a desired effect in a subject being treated. For example, the therapeutically effective amount of the described polymer-nanoparticle composite compound in a solid matrix (such as a bandage) will be the amount necessary to enhance/assist hemostasis and provide antimicrobial effects when brought into contact with a wound.

Hemostasis-promoting polymer: A polymer known in the art to possess hemostasis-promoting properties. One example of a hemostasis-promoting is calcium alginate.

Nanoparticle: A particle with a diameter in the nanometer (nm) range, typically 1 to 1000 nm.

Non-covalent bond: A bond formed between two oppositely charged compounds, but does not involve sharing of one or more electrons between atoms.

Pharmaceutically acceptable carriers: The pharmaceutically acceptable carriers useful in this disclosure are conventional. The Science and Practice of Pharmacy, Adeboye Adejare, Ed., 23rd Edition (2020), describes compositions and formulations suitable for pharmaceutical delivery of the compounds herein disclosed. In general, the nature of the carrier will depend on the particular mode of administration being employed, for example for use as a topical agent in an ointment, cream, or similar suspension.

Subject: Living multi-cellular organisms, including vertebrate organisms, a category that includes both human and non-human mammals.

Under conditions sufficient for [carrying out a desired activity]: A phrase that is used to describe any environment that permits the desired activity.

Wound: An injury to living tissue which can, but does not require breaking skin or bleeding. Particular non-limiting examples of wounds include bruises, burns, and cuts (of varying depths and severity). Wounds can be unintentional, such as resulting from a fall, but can also be intentional, such as a result of surgery or other medical procedure.

Wound dressing: Any covering of any material used to cover a wound. In particular embodiments, wound dressings can be of natural or synthetic fabrics. In other embodiments, wound dressings can be films composed of or including the described compositions. In particular embodiments, a wound dressing does not include any active material. In other embodiments, a wound dressing includes the described compositions, alone, or with other therapeutic agents.

### II. Overview of Several Embodiments

Described herein are compositions for use in methods for treating a wound that include an antimicrobial metal oxide nanoparticle; a first polymer layer coating the antibacterial metal oxide nanoparticle; an external polymer layer coating the polymer-coated nanoparticle; and optionally at least one additional polymer layer between the first polymer layer and the external polymer layer, wherein the first polymer layer is optionally a hemostasis-promoting polymer and/or is loaded with a pharmaceutical agent; and wherein the external polymer layer and the optional at least one additional polymer layer is a hemostasis-promoting polymer, mucoadhesive polymer, and/or is loaded with a pharmaceutical agent that is the same or different from the pharmaceutical agent of the first polymer layer.

In particular embodiments of the described compositions, the antibacterial metal oxide nanoparticle core comprises CuO, ZnO, Ag₂O, TiO₂, MgO, or Fe₂O₃. In other embodiments, the metal oxide nanoparticle core comprises a doped metal oxide, such as but not limited to Zn doped CuO, Cu doped ZnO, Ag doped TiO₂, or Mg doped ZnO.

In particular embodiments of the described composition, metal oxide nanoparticle inhibits bacteria, fungi, and viruses, or a subset of bacteria, fungi, or viruses.

In some embodiments of the described compositions, the first, external, and/or additional polymers are hemostasis-promoting, such as but not limited to calcium alginate, polylysine, oxidized cellulose, chitosan and modifications thereof, gelatin, or thiomers thereof.

In particular embodiments of the described compositions, the pharmaceutical agent is selected from a pain reliever, local anesthetic and/or non-steroidal anti-inflammatory drug (NSAID).

In other particular embodiments, the external polymer layer has a positive or negative zeta potential.

Additionally described herein are wound dressings that include the described compositions for use in treating a wound, either as an intrinsic component of the dressing or as an additive incorporated into or onto the dressing.

Further described herein are topical formulations that include the described compositions, and which can be used directly on the surface of a wound for wound treatment or as an additive onto a wound dressing.

Additionally described herein are methods for treatment of a wound in a subject by administering to the subject a described composition for use in wound treatment.

In particular embodiments of the described methods, the composition is administered to the subject in or on a wound dressing. In other embodiments, the composition is formulated for topical administration and administered directly onto the wound site or added onto a wound dressing which is then applied to the wound site.

### III. Multifunctional Polymer-Nanoparticle Compositions for Wound Care

Described herein is a multi-layered polymer-nanoparticle nanocomposite for use, inter alia, in wound care applications, such as first aid or long-term wound care. The compositions include an antimicrobial metal oxide nanoparticle core, an internal optionally hemostasis-promoting polymer coating on the antibacterial metal oxide nanoparticle core (first polymer coating), and an external hemostatic or mucoadhesive polymer coating. In particular embodiments, the described nanocomposite includes additional polymer layers between the internal polymer coating on the antibacterial metal oxide nanoparticle core and the external hemostasis-promoting or mucoadhesive polymer coating. In some embodiments, one or more of the polymer layers can also be a reservoir for a pharmaceutical agent to be delivered to the wound site. Additionally, although the metal oxide nanoparticle core is coated by at least two polymer layers, these coatings do not entirely interfere with the properties of the metal oxide nanoparticle core, which accordingly retains its antimicrobial functionality.

### Nanoparticle Core

The polymer-nanoparticle nanocomposites described herein contain a metal oxide nanoparticle core with antimicrobial properties. In particular embodiments, the metal oxide is a metal oxide compound containing a single metal species. Non-limiting examples of such metal oxides include CuO, ZnO, Ag₂O, TiO₂, MgO, and Fe₂O₃. In other embodiments, the metal oxides are a “doped” composite including an additional metal. Non-limiting examples of such doped metal oxide composites include Zn doped CuO, Cu doped ZnO, Ag doped TiO₂, and Mg doped ZnO. In a particular example, the metal oxide nanoparticle core is CuO₍₁₋ₓ₎ZnOₓ. Further non-limiting examples of metal oxide composites for use as the nanoparticle core of the described compositions, and methods for their synthesis can be found in U.S. Pat. Nos. 10,995,011 and 10,998,467, the contents of both of which are incorporated by reference herein in their entirety. It will be appreciated however, that any method for producing the described metal oxide nanomaterial known to the art can be used to provide the described antibacterial nanomaterial for use in the disclosed methods.

As noted, the metal oxide nanoparticles for use in the described compositions can be composites of two metal oxides, and is a semiconductor nanomaterial composition that includes metal oxide A and metal oxide B. In a particular embodiment, metal oxide A and metal oxide B are independently selected from a group consisting of zinc (ZnO), copper (CuO), or combinations thereof.

In a particular embodiment, the copper-zinc mixed oxide nanomaterial has a chemical formula of CuO₍₁₋ₓ₎ZnOₓ, wherein x is the atomic ratio of the zinc oxide impurities on the nanomaterial. Generally, the value of x may range from about 0.01 to about 0.26. In various, the value of X may range from about 0.01 to about 0.26, or from about 0.03 to about 0.24. In a preferred embodiment, the value of x may be around 0.2.

As one illustrative example when the nanocomposite is composed of two metal oxides, the metal oxide nanocomposites for use in the described methods can, in particular embodiments, be produced as follows. The process comprises: (a) providing a first aqueous solution comprising a soluble metal salt A and a soluble metal salt B; (b) providing a second aqueous solution comprising at least one soluble anion; (c) admixing the second aqueous solution with the first aqueous solution to form an insoluble precursor metal oxide semiconductor nanomaterial; (d) isolating the metal oxide semiconductor nanomaterial precursor; (e) drying the metal oxide semiconductor precursor; and (f) thermal decomposition of the metal oxide semiconductor precursor to form the metal oxide semiconductor nanomaterial.

### (a) First Aqueous Solution

The process commences by preparing the first aqueous solution comprising a soluble metal salt A and a soluble metal salt B.

As appreciated by the skilled artisan, the soluble metal salts A and B are transformed into metal oxide A and metal oxide B after completion of the process.

In preferred embodiments, soluble metal salt A and soluble metal salt B wherein the metal portion of these salts are independently selected from a group consisting of titanium, silver, magnesium, zinc, copper, or combinations thereof.

A wide variety of anions may be used for soluble metal salt A and soluble metal salt B. An important aspect of these anions is that the anion is readily exchangeable, soluble in aqueous solution, non-toxic, pH neutral, and thermally decomposable. Non-limiting examples of suitable anions may be acetate, propionate, any soluble organic salt or combinations thereof. In a preferred embodiment, the anions used for soluble metal salt A and soluble metal salt B is acetate.

In other embodiments, the first aqueous solution may further comprise one or more different soluble salts than the soluble salts A and soluble salts B as described above.

The molar ratio of the soluble metal salt A to the soluble metal salt B may range from about 12:1 to about 1:12. In various embodiments, the molar ratio of the soluble metal salt A to the soluble metal salt B may range from about 12:1 to about 1:12, from about 11:1 to about 1:11, from about 10:1 to about 1:10, from about 9:1 to about 1:9, from about 8:1 to about 1:8, from about 7:1 to about 1:7, from about 7:1 to about 1:7, from about 6:1 to about 1:6, from about 5:1 to about 1:5, from about 4:1 to about 1:4, from about 3:1 to about 1:3, or from about 2:1 to about 1:2. In a preferred embodiment wherein soluble metal salt A is copper and the soluble metal salt B is zinc, the molar ratio may be about 2.3:1.

In general, the concentration of soluble metal salt A, soluble metal salt B, or combinations thereof in water may range from about 0.01M (moles/liter) to about 1.0M. In various embodiments, the concentration of the soluble metal salt A and soluble metal salt B may range from about 0.01M to about 1.0M, 0.03M to about 0.3M, or from 0.05M to 0.15M. In a preferred embodiment, the concentration of soluble metal salt A, soluble metal salt B, or combinations thereof in water may be about 0.15M.

The first aqueous solution may further comprise a stabilizer. Non-limiting examples of stabilizers may be a polyethylene glycol (PEG), polypropylene glycol (PPG), polyvinylpyrrolidone (PVP), polyvinylalcohol (PVA), Polyoxyethylene or combinations thereof. In a preferred embodiment, the stabilizer used in the first aqueous solution further comprises PEG, specifically PEG4000.

The concentration of the stabilizer in the first aqueous solution may range from about 0.0001M to about 0.001M. In various embodiments, the concentration of the stabilizer in the first aqueous solution may range from about 0.0001M to about 0.001M. In a preferred embodiment, the concentration of the stabilizer in the first aqueous solution may be preferably about 0.0007M.

The preparation of the first solution may be achieved by blending the soluble metal salt A, soluble metal salt B, water, an optional stabilizer, and an optional solvent in any known mixing equipment or reaction vessel until the mixture achieves homogeneity. These components may be added all at the same time, sequentially, or in any order.

In general, the preparation of the first aqueous solution may be conducted at a temperature that ranges from about 10° C. to about 40° C. In various embodiments, the temperature of the reaction may range from about 10° C. to about 40° C., from about 15° C. to about 35° C., or from about 20° C. to about 30° C. In one embodiment, the temperature of the reaction may be about room temperature (˜23° C.). The reaction typically is performed under ambient pressure. The reaction may also be conducted under an inert atmosphere or air, for example under nitrogen, argon or helium.

The duration for preparing the first aqueous solution and will vary depending on many factors, such as the temperature, the method of mixing, and amount of materials being mixed. The duration of the reaction may range from about 5 minutes to about 12 hours. In some embodiments, the duration of the reaction may range from about 5 minutes to about 30 minutes, from about 30 minutes to about 2 hours, from about 2 hours to about 4 hours, from about 4 hours to about 10 hours, or from about 10 hours to about 12 hours. In various embodiments, the preparation may be allowed to continue until the first aqueous solution obtains homogeneity.

### (b) Second Aqueous Solution

The second aqueous solution comprises at least one soluble anion source. An important aspect of these soluble anions is that anion is readily exchangeable, soluble in aqueous solution, is non-toxic, pH neutral, and thermally decomposable. Non-limiting examples of suitable anion sources may be lithium bicarbonate, sodium bicarbonate, potassium bicarbonate, and ammonium bicarbonate, or any alkaline oxalate, alkaline malate. In a preferred embodiment, the second aqueous solution comprises ammonium bicarbonate.

The second aqueous solution may be prepared by forming a reaction mixture comprising at least one soluble anion source, water, and optionally ethanol. These components may be added all at the same time, sequentially, or in any order. The second aqueous solution may be achieved by blending the above components in any known mixing equipment or reaction vessel until the mixture achieves a clear solution.

In general, the preparation of the second aqueous solution may be conducted at a temperature that ranges from about 10° C. to about 40° C. In various embodiments, the temperature of the preparation may range from about 10° C. to about 40° C., from about 15° C. to about 35° C., or from about 20° C. to about 30° C. In one embodiment, the temperature of the preparation may be about room temperature (˜23° C.). The preparation typically is performed under ambient pressure. The preparation may also be conducted under air or an inert atmosphere, for example under nitrogen, argon or helium.

The duration for preparing the second aqueous solution and will vary depending on many factors, such as the temperature, the method of mixing, and amount of the at least one anion source being mixed. The duration of the reaction may range from about 5 minutes to about 12 hours. In some embodiments, the duration of the reaction may range from about 5 minutes to about 30 minutes, from about 30 minutes to about 2 hours, from about 2 hours to about 4 hours, from about 4 hours to about 10 hours, or from about 10 hours to about 12 hours.

Generally, the concentration of the at least one soluble anion source in the second aqueous solution may range from a concentration of about 0.10M to about 1.5M. In various embodiments, the concentration of the at least one soluble anion source in the second aqueous solution may range in a concentration from about 0.10M to about 1.5M, from about 0.2M to about 1.4M, or from about 0.3M to about 1.2M. In a preferred embodiment, the concentration of the at least one soluble anion source in the second aqueous solution may be about 0.3M.

### (c) Preparation of the Insoluble Metal Oxide Semiconductor Nanomaterial Precursor.

The next step in the process is to prepare the insoluble metal oxide semiconductor nanomaterial precursor. Preparing the insoluble metal oxide semiconductor nanomaterial precursor occurs when the second aqueous solution comprising the at least one anion source is admixed with the first aqueous solution. As appreciated by the skilled artisan, once the second aqueous solution is admixed with the first aqueous solution, a chemical reaction occurs. In a particular illustrative embodiment, the metal oxide semiconductor nanomaterial precursor comprising a copper zinc mixed carbonates are formed and can be depicted according to the following scheme.

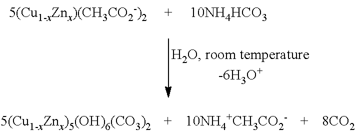

As appreciated by the skilled artisan, an advantage of using ammonium salt in the second aqueous solution is that by product, ammonium acetate, is water soluble, easily removed from the metal oxide semiconductor nanomaterial precursor, neutral pH at room temperature, and trace amount of ammonium acetate are readily thermally decomposed in the process.

The process may further comprise an organic solvent. The purpose of the solvent in the process is to reduce the foaming as the two aqueous solutions are combined, namely carbon dioxide. The addition of solvent may also cause a sudden change of the dielectric constant and change the dynamic of precipitation of the insoluble metal oxide semiconductor nanomaterial precursor. These changes may further lead to a hierarchic structure, a core-shell configuration of the metal oxide semiconductor nanomaterial, or combinations of both of properties. An additional property of the solvent is that solvent is volatile so excess amounts of solvent may be readily removed. Non-limiting examples of suitable solvents may be methanol, ethanol, propanol, iso-propanol, acetone or combinations thereof. In a preferred embodiment, the solvent in the process is ethanol.

Generally, the volume percent of the solvent in the mixture of the first aqueous solution, the second aqueous solution or combinations thereof may range from about 0.01 volume % to about 0.1 volume % In various embodiments, the volume percent of the solvent in the mixture of the first aqueous solution, the second aqueous solution or combinations thereof may range from about 0.01 volume % to about 0.1 volume %, from about 0.02 volume % to about 0.08 volume %, or from about 0.03 volume % to about 0.07 volume %. In a preferred embodiment, the volume percent of the solvent in the mixture of the first aqueous solution, the second aqueous solution or combinations thereof may be about 0.02 volume %.

The solvent may be added to the first aqueous solution, the second aqueous solution, or the combination of the first aqueous solvent and the second aqueous solvent, or combinations thereof.

The metal oxide semiconductor nanomaterial precursor may be prepared by forming a reaction mixture comprising the first aqueous solution, the second aqueous solution, and the optional solvent. The metal oxide semiconductor nanomaterial precursor may be achieved by blending the above components in any known mixing equipment or reaction vessel or static mixer until the mixture achieves completeness of reaction.

In an embodiment, the second aqueous solution may be added to the first solution. Generally, the second aqueous solution is added immediately in a batch or by a static mixer continuously in a range from about 20 volume % to about 45 volume % to the first aqueous solution. In a speed from 1 to 10 l/min, in various embodiments from 1.25 to 8 l/min. In a preferred embodiment 5 to 6 l/min. This quick addition ensures the chemical reaction depicted above goes to completion.

Since the insoluble metal oxide semiconductor nanomaterial precursor precipitates from an aqueous solution, the method of stirring to prepare the precursor is important so amounts of the soluble metal salt A, metal salt B, or the at least one soluble anion source does not become entrained in the insoluble metal oxide semiconductor nanomaterial precursor. Generally, the process may be stirred mechanically at a speed from about 250 rpm (revolution per minute) to about 1000 rpm. In various embodiments, the stirring speed may range from 250 rpm to about 1200 rpm, from about 300 rpm to about 1000 rpm, or from about 500 rpm to about 900 rpm. In a preferred embodiment, the stirring speed of the process may be about 700 rpm.

In general, the preparation of the insoluble metal oxide semiconductor nanomaterial precursor may be conducted at a temperature that ranges from about 10° C. to about 65° C. In various embodiments, the temperature of the preparation may range from about 10° C. to about 65° C., from about 15° C. to about 35° C., or from about 20° C. to about 30° C. In one embodiment, the temperature of the preparation may be about room temperature (˜23° C.). The preparation typically is performed under ambient pressure. The preparation may also be conducted under air or an inert atmosphere, for example under nitrogen, argon or helium.

The pH during the addition of the reaction between the second aqueous solution and the first aqueous solution may range from about 6.0 to about 8.0. In various embodiments, the pH of the process may range from about 6.0 to about 8.0, from about 6.5 to about 7.5, or from about 6.7 to about 7.3. In a preferred embodiment, the pH of the process is about 6.8 to 7.0.

The duration for preparing the insoluble metal oxide semiconductor nanomaterial precursor and will vary depending on many factors, such as the temperature, the method of mixing, and scale of the process. The duration of the reaction may range from about 5 minutes to about 6 hours. In some embodiments, the duration of the reaction may range from about 5 minutes to about 6 hours, from about 15 minutes to about 4 hours, or from about 20 minutes to about 1 hour. In a preferred embodiment, the duration for preparing the insoluble metal oxide semiconductor precursor may be about 30 minutes.

### (d) Isolating the Insoluble Metal Oxide Semiconductor Nanomaterial Precursor

The next step in the process is isolating the insoluble metal oxide semiconductor nanomaterial precursor from the reaction mixture in step (c) comprising water, the stabilizer, and the optional solvent. As appreciated by the skilled artisan, there are many methods of isolating the insoluble metal oxide semiconductor nanomaterial precursor from the reaction mixture in step (c). Non-limiting methods may be filtration, centrifugal separation, decantation, or combinations thereof. The insoluble metal oxide semiconductor nanomaterial precursor, after isolation, may be rinsed with water, ethanol, or combinations thereof. The precursor is washed with water, ethanol, or combinations thereof solvent until the supernatant is colorless or the precursor color remains constant.

### (e) Drying the Insoluble Metal Oxide Semiconductor Precursor.

The next step in the process is drying the insoluble metal oxide semiconductor nanomaterial precursor from the reaction mixture in step (d). This step would remove excess amounts of solvent from the insoluble metal oxide semiconductor nanomaterial precursor. As appreciated by the skilled artisan, many devices are available to dry the precursor. Non-limiting examples for drying the solid may be batch driers, convection ovens, rotary dryers, drum dryers, kiln dryers, flash dryers, or tunnel dryers.

In general, the drying of the insoluble metal oxide semiconductor nanomaterial precursor may be conducted at a temperature that ranges from about 30° C. to about 120° C. In various embodiments, the temperature of the preparation may range from about 30° C. to about 120° C., from about 40° C. to about 100° C., or from about 50° C. to about 80° C. In one embodiment, the temperature of drying may be about 60° C. The preparation typically is performed under ambient pressure. The preparation may also be conducted under air or an inert atmosphere, for example under nitrogen, argon or helium.

The duration for drying the insoluble metal oxide semiconductor nanomaterial precursor and will vary depending on many factors, such as the temperature, the amount of the precursor, and type of the dryer. The duration of the reaction may range from about 30 minutes to about 48 hours. In some embodiments, the duration of the reaction may range from about 30 minutes to about 48 hours, from about 1 hour to about 24 hours, or from about 2 hours to about 4 hours. In a preferred embodiment, the duration for drying the insoluble metal oxide semiconductor precursor may be about 3 hours, or until the drying the insoluble metal oxide semiconductor precursor reaches less than 12% moisture.

### (f) Thermal Decomposition of the Insoluble Metal Oxide Semiconductor Nanomaterial Precursor Forming the Metal Oxide Semiconductor Nanomaterial

The next step in the process is thermal decomposition of the insoluble metal oxide semiconductor nanomaterial precursor forming the metal oxide semiconductor nanomaterial. This step removes transforms the thermally labile ligand forming the oxides and removes by-products and impurities that were not removed in step (d). As appreciated by the skilled artisan, carbon, hydrogen and excessive oxygen may be released in forms of carbon dioxide and water steam from the thermally labile ligands, by-products, and impurities. In a preferred embodiment, the metal oxide semiconductor nanomaterial precursor comprising a copper zinc mixed oxide is thermally decomposed to form the metal oxide semiconductor nanomaterial. This reaction can be depicted according to the following scheme.

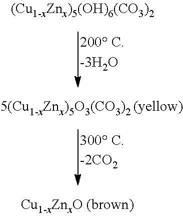

In general, thermal decomposition of the insoluble metal oxide semiconductor nanomaterial precursor may be conducted at a temperature that ranges from about 200° C. to about 1000° C. In various embodiments, the temperature of the preparation may range from about 200° C. to about 1000° C., from about 225° C. to about 800° C., or from about 250° C. to about 350° C. In one embodiment, the temperature of drying may be about 300° C. The preparation typically is performed under ambient pressure. The preparation may also be conducted under air or an inert atmosphere, for example under nitrogen, argon or helium.

The duration for drying the insoluble metal oxide semiconductor nanomaterial precursor and will vary depending on many factors, such as the temperature, the amount of the precursor, and type of the dryer. The duration of the reaction may range from about 5 minutes to about 48 hours. In some embodiments, the duration of the reaction may range from about 10 minutes to about 48 hours, from about 15 hour to about 24 hours, or from about 2 hours to about 4 hours. In a preferred embodiment, the duration for drying the insoluble metal oxide semiconductor precursor may be about 0.3 hour.

The yield of the metal oxide semiconductor material from the process described above may range from 5 to 12 g/L. with high purity.

The diameter of the metal oxide nanoparticle core in the described compositions can vary, but will be in the nanometer range in at least one dimension. For example, the size of at least one dimension of metal oxide nanoparticle may range from about 1 nanometers (nm) to 1,000 nm, such as from about 10 nm to 1,000 nm, from about 10 nm to about 500 nm, from about 100 nm to about 1,000 nm, and from about 10 nm to about 150 nm. In particular embodiments, the core is from about 10 nm to about 250 nm.

The nanoparticle/nanocomposite core for use in the described compositions is not limited by shape. In particular embodiments, nanoparticle/nanocomposite core can be a sphere, plate, rod, and/or polyhedron.

The metal oxide nanoparticle core of the described compositions is antimicrobial. As used herein, the term antimicrobial encompasses inhibition or eradication of both living microbes (e.g., bacteria and fungi) and non-living microbes (e.g., viruses). In particular embodiments the metal oxide nanoparticle core is generally inhibitory against microbial growth and reproduction, and can inhibit bacteria, fungi, and viruses. In another embodiment, the metal oxide nanoparticle core selectively inhibits one or more particular class or species of microbe.

### Polymer Coatings

The described polymer-nanoparticle composite compositions include at least two polymer layers coating the metal oxide nanoparticle core. The first polymer layer directly coats the metal oxide core, and is also described herein as the internal layer. The second polymer layer is an external (i.e., surface exposed) polymer layer that coats the entire composite composition. Particular embodiments of the described composite compositions include at least one additional polymer layer between the first polymer layer and the external polymer layer, such that the final number of polymer layers in the composite composition can be 2, 3, 4, 5, 6, 7, 8, 9, 10, or even more polymer layers.

Non-limiting examples of the polymers for use in the described compositions include an alginate salt such as calcium alginate or sodium alginate, polylysine, oxidized cellulose, chitosan and its modifications such as but not limited to chitosan mesylate salts, chitosan HCl salt, chitosan catechol, carboxymethyl chitosan, thiolated chitosan, acrylated chitosan and the like, gelatin, and thiomers of the foregoing polymers. Each of the polymer layers in the described composite composition is independent of each other polymer layer. Additionally, each polymer layer can provide a separate functionality to the described polymer-nanoparticle composite. In a particular embodiment the first polymer layer can be a known hemostasis-promoting polymer, and the external polymer has an application-specific zeta potential, allowing for effective non-covalent interactions with negatively charged surfaces or positively charged surfaces as required. Further, while each polymer layer is noted as “coating” the metal oxide nanoparticle core or the other polymer layers beneath it, the polymer layers do not completely inhibit the antimicrobial properties of the metal oxide nanoparticle core.

In particular embodiments, the first polymer layer is a known hemostasis-promoting polymer and the external polymer layer is mucoadhesive. In other particular embodiments, the first polymer layer is hemostasis-promoting and the external polymer layer is hemostasis-promoting and optionally mucoadhesive. In another embodiment, the first polymer layer is not hemostasis-promoting. In a specific embodiment, first polymer layer is calcium alginate and the external polymer layer is modified chitosan. If yet another specific embodiments, the described composite compositions have 2, 3, 4, 5 or more layers where the first layer is an alginate such as calcium alginate, and the second layer is a chitosan or modification thereof, and the remaining layers alternate between alginate and chitosan. In other particular embodiments, a chitosan or modification thereof can be the first polymer layer, followed by an alginate in the second polymer layer.

In additional embodiments, one or more of the polymer layers is loaded with an active pharmaceutical compound. Particular non-limiting examples of pharmaceutical compounds that can be included in one or more of the polymer layers include an analgesic compound, local anesthetic, anti-inflammatory agent, non-steroidal anti-inflammatory drug (NSAID) (which can combine analgesia and anti-inflammatory functionalities), and/or a topical antibiotic. Non-limiting examples of analgesic compounds include aspirin, acetaminophen, opiates, and derivatives thereof. Non-limiting examples of anesthetics include tetracaine, procaine, lidocaine, and borneol. Non-limiting examples of NSAIDs include Ibuprofen, diclofenac, ketorolac, and meloxicam. Non-limiting examples of antibiotics include β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim.

As noted, each layer of the composite composition is independent of each subsequent layer, with the exception being that the layering must be chemically (electrostatically) compatible. Thus, in particular embodiments, a multilayered composite composition has alternating layers of the same polymer pair (e.g., calcium alginate and chitosan), whereas in other embodiments the multilayered composite composition is layered with different electrostatically-compatible polymers.

Similarly, because each polymer layer can be loaded with a different therapeutic compound, particular embodiments of the described composite compositions can provide different therapeutic functionalities to a subject in differently loaded polymers. For example, in one embodiment, the described compositions can provide an analgesic in one or more layers. In another embodiment, the described compositions can provide an analgesic in one or more layers and an anti-inflammatory drug in one or more other layers. In yet another embodiment, the described compositions can provide analgesia, anti-inflammatory properties, and antibiotic therapeutic effects.

### Wound Care

The polymer-nanoparticle composite described herein provides a multifunctional composition for use in wound care. As noted, the described composites combine at least antimicrobial and drug release efficacy from the metal oxide core and first polymer layer, respectively. The first polymer can also promote or enhance hemostasis depending on the known characteristics of the first polymer (e.g., in particular embodiments, the first polymer is calcium alginate, a polymer known to promote hemostasis). Additional functionality, such as mucoadhesion and/or an additional hemostatic effect is provided by the external polymer layer and other optional polymer layers between the first polymer layer and the external polymer layer. In addition to the functionalities provided by the metal oxide nanoparticle core and surrounding polymer layers, the described composition can include one or more pharmaceutical agents to provide pain relief, local anesthesia, additional antibiotic, anti-inflammatory effects and the like. The described compositions can therefore be used for multifunctional treatment of a wound in a subject.

In particular embodiments, the wound treatment is in the context of first aid, directly following injury to the subject. In other embodiments, the treatment is provided for long-term wound care. Wounds that can be treated with the described compositions include both external (e.g., surface) wounds, but also can be internal (e.g., from an internal incision following a surgical procedure). External wounds can include those in which the skin surface is broken, including small and large cuts, abrasions, and the like. External wounds can also include burns, blisters, and similar lesions.

In particular embodiments, the described compositions are applied to and/or incorporated into the fibers of a wound dressing that is then provided to a subject in need thereof. The wound dressing can be of any material, including any material composed a sorted matrix of fibers, including cotton textiles, gauze, and electrospun non-woven fabrics. The wound dressings for use in the current treatments can carry a net negative or net positive charge at the surface. Accordingly, particular embodiments of the described compositions which have a positive zeta potential from the external polymer layer (e.g., chitosan), can be stably associated with negatively-charged wound dressings by way of electrostatic interaction between the polymer-nanoparticle composite and the wound dressing material. Conversely, in other embodiments, the net zeta potential of the external polymer layer is negative, enabling its stable association with wound dressing materials having a net positive charge.

In other embodiments, the wound dressing itself is composed of the described polymer-nanoparticle composite composition. In one non-limiting example, the polymer-nanoparticle composite can be formed into a film which can be further processed into a wound dressing.

In other embodiments, the polymer-nanoparticle composite composition is formulated in a spreadable formulation such as a cream, suspension, or ointment that can be applied to the wound surface and which is then covered by a wound dressing. Alternatively, the spreadable formulation is applied to the wound dressing which is then used to cover the wound. In still further embodiments, the polymer-nanoparticle composite composition is formulated for administration to varied external and internal surfaces. The described formulations include therapeutically effective amounts of the described polymer-nanoparticle composite composition in lotions, foams, patches, gels, suspensions, and solutions, all of which can be prepared by standard methods of the art.

The polymer-nanoparticle composite compositions described herein are provided in methods for treating a wound by administering the composition to a subject in need of such treatment. In particular embodiments, the compositions are administered as part of a first aid treatment, such as in the first minutes or hour following an injury. In other embodiments, the compositions are administered as part of a long or longer-term wound care treatment, such as following initial treatment of a wound. The multifunctional properties of the described compositions inhibit microbial growth at the wound site, and aid in cessation of blood flow, thereby improving wound healing. As noted, the addition of pain relief, anti-inflammation, and additional antibiotic functionalities by inclusion of pharmaceutical agents in the polymer layers combine to further improve the wound healing capabilities of the described compositions.

One particular embodiment of the described compositions for use in methods of wound care includes a polymer-nanoparticle composite that includes a CuO₍₁₋ₓ₎ZnOₓ metal oxide core, a first polymer layer of calcium alginate, and which is optionally loaded with a local anesthetic, and an external layer of inorganic acid-modified chitosan. In this embodiment, the alginate and/or the chitosan polymer can be loaded with a therapeutic agent prior to incorporation within the composite. Further variations on this embodiment include multiple alternating layers of alginate and chitosan, with chitosan being the external layer. In a variation on the noted embodiment, instead of the first polymer layer being composed of calcium alginate, it is composed of modified chitosan, and the second polymer layer is composed of calcium alginate. In such embodiments wherein the composite composition is intended for use in certain wound dressing material, the external layer will be chitosan, in order to provide the net external electrostatic charge that is compatible with gauze or other wound dressing material that has a net negative charge.

### IV. Methods of Manufacturing a Multifunctional Polymer-Nanoparticle Composition for Wound Care

Particular embodiments of the polymer-nanoparticle composites described herein are produced in a scalable process as follows. The possible components of the metal oxide nanoparticle core and coating polymers in this embodiment are as described above. As described above, the first polymer is optionally a polymer that is known to promote or enhance hemostasis and/or has been loaded with an additional pharmaceutical agent, while the external polymer may have a positive zeta potential or a negative zeta potential.

In general, the preparation method can be conducted at a temperature that ranges from about 10° C. to about 40° C., such as from about 15° C. to about 35° C., from about 20° C. to about 30° C. In one embodiment, the temperature of the reaction may be about room temperature (˜25° C.), unless otherwise specified.

In a first step, a metal oxide nanoparticle is provided in an aqueous suspension. To this suspension is added an aqueous solution containing an aqueous-soluble polymer having known hemostatic enhancing properties, or a precursor thereof (“the first polymer”). The mass ratio of polymer to the nanoparticle is between the range of 1:1 and 1:10. This mixture is mixed 0.5 to 18 hours, such as 12 hours. Excess polymer is separated from the polymer-coated nanoparticle by any suitable method, such as centrifugation.

In particular embodiments the first polymer does not intrinsically enhance or promote hemostasis. Hemostasis-promoting/enhancing functionality is added by ion exchange according to standard methods. In a particular embodiment the first polymer is a sodium or other non-calcium salt of alginate, such as sodium alginate. In such embodiments, the sodium is exchanged for calcium by ion exchange for sufficient time by mixing, for example, with calcium chloride. The result of the ion exchange is a hemostasis-promoting/enhancing first polymer-coated nanoparticle. As with the first step, the polymer-coated nanoparticle is separated from the excess calcium salt by standard methods, such as centrifugation.

In particular embodiments, one or more additional polymer layers is added prior to the “final” or external polymer layer. Such additional layering is done by sequential steps of mixing and separation of oppositely charged polymer layers one on top of the other. For each possible layering step, the mass ratio of polymer to the nanoparticle is between the range of 1:1 and 1:10.

The external layer of the polymer-nanoparticle composite is a polymer having a positive or negative zeta potential, depending on the desired downstream application, and is layered on top of the prior layers as described. However, in particular embodiments, the external polymer requires modification prior to being combined with the polymer-nanoparticle composite to increase its aqueous solubility, thereby allowing for its addition to the described composite in aqueous suspension. In a particular example, chitosan is the external polymer and is modified by reaction with an organic or inorganic acid, such as methane sulfonic acid or HCl. Excess acid is removed from the modified polymer by dialysis.

The following examples are provided to illustrate certain particular features and/or embodiments. These examples should not be construed to limit the disclosure to the particular features or embodiments described.

### EXAMPLES

### Example 1: Preparation of Multi-Functional Polymer-Nanoparticle Composite with Antibacterial and Hemostatic Properties

This example describes a method for manufacturing a polymer-nanoparticle composite composition, which in turn can be used in a range of medical applications. The produced composition includes an antibacterial nanoparticle core, a first, core-proximal polymer layer, and a second, surface exposed polymer layer. The method is carried out according the to the following steps:

Step 1, coating the nanoparticle with a first polymer layer: To an antibacterial nanocomposite suspension in deionized water (CuO₍₁₋ₓ₎ZnOₓ at 7.5 g/L, prepared according to US Patent Publication No. 2020/0231459) was added an aqueous solution of Na-Alginate (2.25 g/L) dropwise while stirring. The solution was stirred for 12 h at room temperature. Excess/unbound polymer was removed by centrifugation to obtain a Na-Alginate coated nanocomposite slurry.

Step 2, ion exchange: To prepare Ca-Alginate coated nanocomposite, an ion exchange reaction was performed. To the suspension of Na-alginate coated nanocomposite, aq. CaCl₂ (10 g/L) was added dropwise and stirred for 3 h. Excess CaCl₂ was removed by centrifugation to get Ca-Alginate coated nanoparticles slurry.

Step 3, coating the first nanoparticle-polymer composite with a second polymer layer: To add a layer of oppositely charged polymer surrounding the particle-proximal layer, first a water-soluble modified chitosan was prepared. Chitosan (1 g) was suspended in water (80 ml) at −10° C. To this, methane sulfonic acid or HCl (˜1 mL) was added dropwise until the solution became clear, and was stirred for an additional hour. Resulting modified chitosan was purified by 48 h dialysis against deionized water.

To the suspension of Ca-alginate coated nanocomposite in deionized water (7.5 g/L), an aqueous solution of the modified chitosan (2.25 g/L) was added while stirring. The solution was stirred for 8 h at room temperature. Excess/unbound polymer was removed by centrifugation and the slurry was dried under vacuum to obtain chitosan/Ca-alginate coated nanocomposite.

### Characterization of Multifunctional Nanocomposite

The nanocomposite generated by the described method was characterized by assaying the surface zeta potential and the amount of polymer on the generated material.

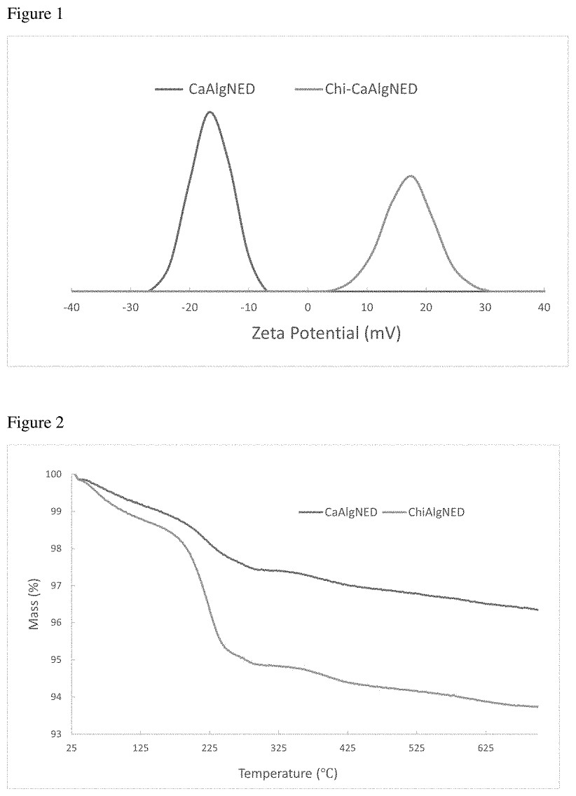

The surface potential of the nanoparticles is modified when coated with different polyelectrolytes. As shown in FIG. 1, there was a clear correlation between the observed surface potential of the nanoparticles to the nature of coated polyelectrolytes. For calcium alginate- (CaAlginate) coated nanoparticles, the surface potential was −16 mV, and this value shifts to +16 mV when the layer of chitosan was added over alginate layer.

Additionally, Thermogravimetric analysis (TGA) was used to determine mass percentage of different polymers in the final composite was obtained, and is shown in FIG. 2. A gradual increase in the mass percentage from −2% in the calcium alginate-coated nanoparticles (CaAlgNED) to ˜5% in chitosan/alginate-coated nanoparticles (ChiAlgNED) indicates layer-by-layer deposition of polymers.

### Example 2: Preparation of Multi-Functional, Drug-Releasing, Polymer-Nanoparticle Composite with Antibacterial and Hemostatic Properties

This example describes preparation of the composite composition of Example 1, but which contains anesthetic-loaded first polymer.

Step 1, coating the nanoparticle with a drug-loaded first polymer layer: Tetracaine (20 mg) was dissolved in deionized water (20 mL), added to a sodium alginate solution (75 mL, 2.667 g/L), and stirred for 3 h. The resulting tetracaine-loaded sodium alginate was added dropwise to an antibacterial nanocomposite (200 mL, 10 g/L), and stirred overnight. The suspension was centrifuged twice at 10000 rpm, for 5 min to remove excess of tetracaine and sodium alginate. Resulting Tetracaine-loaded Alginate coated nanocomposite was either stored as concentrated suspension or dried powder for further use. Prior to use in Step 2 below, the tetracaine-loaded sodium alginate coated nanocomposite was converted to tetracaine-loaded calcium alginate coated nanocomposite by ion exchange as in Example 1.

The actual amount of loaded tetracaine drug in the Tetracaine-loaded Alginate coated nanocomposite was determined against an appropriate calibration curve using UV-Vis spectroscopy, determined to be around 16.8 micrograms per milligram of material.

Step 2, coating the first nanoparticle-polymer composite with a second polymer layer: To add a layer of oppositely charged polymer surrounding the particle-proximal layer, first a water-soluble modified chitosan was prepared. Chitosan (1 g) was suspended in water (80 ml) at ˜10° C. To this, methane sulfonic acid or HCl (˜1 mL) was added dropwise until the solution became clear, and was stirred for an additional hour. Resulting modified chitosan was purified by 48 h dialysis against deionized water.

In the next step, modified chitosan was coated over the Tetracaine loaded Alginate coated nanocomposite. To a suspension of tetracaine loaded alginate coated nanocomposite in deionized water (7.5 g/L), an aqueous solution of the modified chitosan (2.25 g/L) was added while stirring. The solution was stirred for 8 h at room temperature. Excess/unbound polymer was removed by centrifugation and the slurry was dried under vacuum to obtain tetracaine chitosan/alginate coated nanocomposite.

### Example 3: Preparation of Multi-Functional Polymer-Nanoparticle Composite with Antibacterial and Hemostatic Promoting Properties with Five Layers

This example describes a method for manufacturing a polymer-nanoparticle composite composition, which in turn can be used in a range of medical applications for example as described herein. The produced composition includes an antibacterial nanoparticle core and layer-by-layer alternative deposition of sodium alginate and chitosan polymers up to five layers. The method is carried out according the to the following steps:

Step 1: Coating the nanoparticle with a first polymer layer. To an antibacterial nanocomposite suspension in deionized water (CuO₍₁₋ₓ₎ZnOₓ at 7.5 g/L, prepared according to US Patent Publication No. 2020/0231459), and as described above, was added an aqueous solution of Na-Alginate (2.25 g/L) dropwise while stirring. The solution was stirred between 4 to 12 hours at room temperature. Excess/unbound polymer was removed by centrifugation to obtain a Na-Alginate coated nanocomposite slurry.

Step 2: To the suspension of Na-alginate coated nanocomposite in deionized water (7.5 g/L), an aqueous solution of the modified chitosan (2.25 g/L) was added while stirring. The solution was stirred for 4 to 12 hours at room temperature. Excess/unbound polymer was removed by centrifugation and the slurry was dried under vacuum to obtain chitosan/Na-alginate coated nanocomposite.

Steps 1 and 2 were repeated until antibacterial nanoparticle core was coated with five alternating layers of Na-Alginate and chitosan. Chitosan is a hemostasis promoting polymer.

### Characterization of Multifunctional Nanocomposite

The nanocomposite generated by the described method was characterized by assaying the surface zeta potential and the amount of polymer on the generated material.

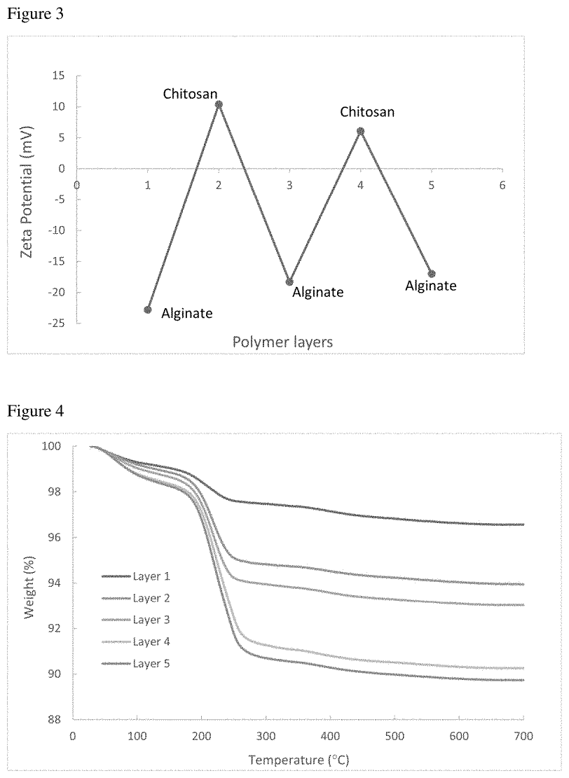

The surface potential of the nanoparticles is modified when coated with different polyelectrolytes. As shown in FIG. 3, there was a clear correlation between the observed surface potential of the nanoparticles to the nature of coated polyelectrolytes. For sodium alginate- (Na-Alginate) coated nanoparticles, a negative surface potential was observed, and this value shifts to positive potential when the layer of chitosan was added over alginate layer.

Additionally, thermogravimetric analysis (TGA) was used to determine mass percentage of different polymers in the final composite was obtained and is shown in FIG. 4. As shown in the figure, a gradual increase in the mass percentage with the addition of each polymer layer indicates layer-by-layer deposition of polymers.

### Example 4: Preparation of Multi-Functional, Ibuprofen-Releasing, Polymer-Nanoparticle Composite with Antibacterial and Hemostasis Promoting Properties

This example describes preparation of a two-layer composite composition which contains Ibuprofen, representing a typical acidic anesthetic drug, loaded into the second polymer layer.

Step 1, coating the nanoparticle with first polymer layer Na-Alginate was performed as in Example 5.

Step 2: coating the first nanoparticle-polymer composite with a drug-loaded second polymer layer. Ibuprofen (20 mg) was first dissolved in aqueous alkaline solution (20 mL deionized water+30 drops of 0.1 M NaOH) and stirred overnight. Ibuprofen solution was then added to the modified water-soluble chitosan solution, prepared as in the preceding examples (75 mL, 2.667 g/L), and stirred for 3 hours. Resulting Ibuprofen loaded chitosan was added dropwise to alginate coated nanocomposite (300 mL, 6.667 g/L) and stirred overnight. Suspension was centrifuged at 10000 rpm, 5 min to remove excess Ibuprofen and chitosan.

The antimicrobial effect was determined according to the ASTM International standard assessment of antimicrobial activity using a time-kill procedure. (available online at astm.org/cgi-bin/resolver.cgi?E2315-16). Briefly, after 2 hours of preconditioning using 5% FBS, 0.5 ml bacterial suspension (2×10⁸) was mixed into the noted amount of the nanocomposite polymer composition and the test tube was incubated at 30° C. for 2 hours. At the end of incubation, a sample was taken and serially diluted and plated.

At 20 ppm concentration, 90 min contact time, bactericidal activity against Escherichia coli was 99.80%.

The drug loading efficiency was determined by analyzing the UV-Vis spectrum of the supernatant. Drug loading efficiency (%)=(Amount of drug loaded/Total amount of drug used)×100. The drug loading efficiency of the polymer nanoparticle composite was approximately 41%.

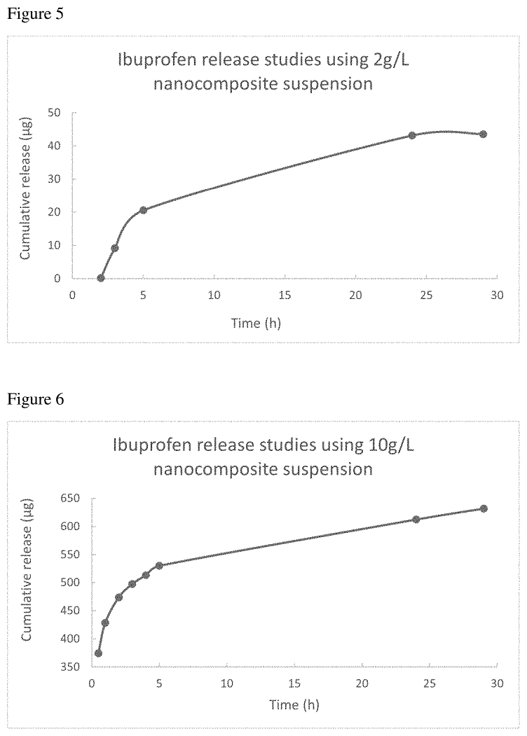

Release of ibuprofen from polymer-nanoparticle composite was done in pH 7.4 phosphate buffer medium. 10 mL of multifuctional nanocomposite of concentrations 2 g/L and 10 g/L was dialyzed against phosphate buffer (100 mL). Nanocomposite concentration can be chosen according to the required amount of drug release. UV-Vis spectrum of the buffer was analyzed at different time intervals to determine the drug release kinetics. Drug release profiles obtained 37° C. are presented in FIG. 5 (2 g/L) and FIG. 6 (10 g/L).

### Example 5: Preparation of Multi-Functional, Tetracaine-Releasing, Polymer-Nanoparticle Composite with Antibacterial and Hemostasis Promoting Properties

This example describes preparation of the composite composition which contains Tetracaine, representing a typical basic anesthetic drug, loaded into the second polymer, which in this example is net negatively charged.

Step 1, coating the nanoparticle with first polymer layer chitosan was performed as in previous examples.

Step 2, coating the first nanoparticle-polymer composite with a drug-loaded second polymer layer. Tetracaine (20 mg) was dissolved in deionized water (20 mL), added to a sodium alginate solution (75 mL, 2.667 g/L), and stirred for 3 hours. The resulting tetracaine-loaded sodium alginate was added dropwise to the chitosan coated antibacterial nanocomposite (200 mL, 10 g/L), and stirred overnight. The suspension was centrifuged at 10000 rpm, for 5 minutes to remove excess of tetracaine and sodium alginate.

Bactericidal activity was determined as described in the preceding examples. At 20 ppm concentration, 90 minutes of contact time, bactericidal activity against Escherichia coli was 99.27%.

The drug loading efficiency was determined by analyzing the UV-Vis spectrum of the supernatant. Drug loading efficiency (%)=(Amount of drug loaded/Total amount of drug used)×100. The drug loading efficiency of the polymer nanoparticle composite was in the range of 60 to 70%.

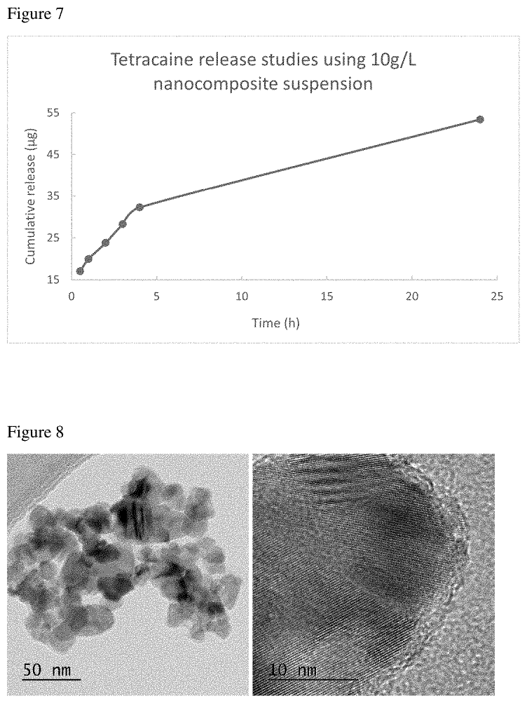

Release of tetracaine from polymer-nanoparticle composite was done in pH 7.4 phosphate buffer medium. 10 mL of multifuctional nanocomposite of concentrations 10 g/L was dialyzed against phosphate buffer (100 mL). Nanocomposite concentration can be chosen according to the required amount of drug release. UV-Vis spectrum of the buffer was analyzed at different time intervals to determine the drug release kinetics. The drug release profile obtained at 37° C. is presented in FIG. 7.

### Example 6: Preparation of Multi-Functional, Multilayer Tetracaine-Releasing, Polymer-Nanoparticle Composite with Antibacterial and Hemostasis Promoting Properties

This example describes preparation of an embodiment of the described composite composition which contains an anesthetic drug Tetracaine in two layers of a four-polymer layer coated nanocomposite. This multifunctional nanocomposite was prepared to increase the amount of drug loading and to enhance overall hemostasis promoting ability.

Step 1, coating the nanoparticle with a first polymer layer of chitosan was performed as in previous examples.

Step 2, coating the nanoparticle-chitosan composite with a tetracaine-loaded second layer of Na-Alginate was performed as in previous examples.

Steps 1 and 2 were then repeated until the antibacterial nanoparticle core was coated with four alternating layers of chitosan and tetracaine-loaded Na-Alginate. Chitosan is a hemostasis promoting polymer. High resolution transmission electron microscopy (HRTEM) images of the resultant multifunctional four polymer layer nanocomposite are shown in FIG. 8.

The drug loading efficiency of the polymer nanoparticle composite was determined as previously performed and was approximately 60%.

Bactericidal efficacy of the multi-layered polymer nanoparticle composites loaded with Tetracaine was tested as in the preceding examples, against a spectrum of Gram (+Ve) and Gram (−Ve) bacteria. For comparison, bactericidal efficacy was compared with a composition of only the nanoparticle material (NED: CuO₍₁₋ₓ₎ZnOₓ). As shown in the following table, the bactericidal properties of the nanocomposite-polymer composition were unchanged with addition of the chitosan and alginate polymer layers.

| | Bactericidal effect (% Killing), 100 ppm concentration | | | |
|---|---|---|---|---|
| | Escherichia | Klebsiella | Staphylococcus | Listeria |
| Sample | coli | pneumoniae | aureus | monocytogenes |
| NED | 86.58 | 99.96 | 99.9 | 99.99 |
| AlgChiAlgChiNED | 90.09 | 99.999 | 99.98 | 99.90 |

### Example 7: Preparation of Multi-Functional, Multilayer Ibuprofen-Releasing, Polymer-Nanoparticle Composite with Antibacterial and Hemostasis Promoting Properties

This example describes preparation of the composite composition which contains an anesthetic drug Ibuprofen in two layers of a total of a four-polymer layer coating. This multifunctional nanocomposite was prepared to increase the amount of drug loading and to enhance overall hemostasis promoting ability.

Step 1, coating the nanoparticle with first polymer layer Na-Alginate was performed as in previous examples.

Step 2, coating the first nanoparticle-polymer composite with Ibuprofen-loaded second polymer layer chitosan was performed as in previous examples.

Steps 1 and 2 were repeated until antibacterial nanoparticle core is coated with four alternating layers of Na-Alginate and Ibuprofen-loaded chitosan.

The drug loading efficiency of the polymer nanoparticle composite was approximately 28%.

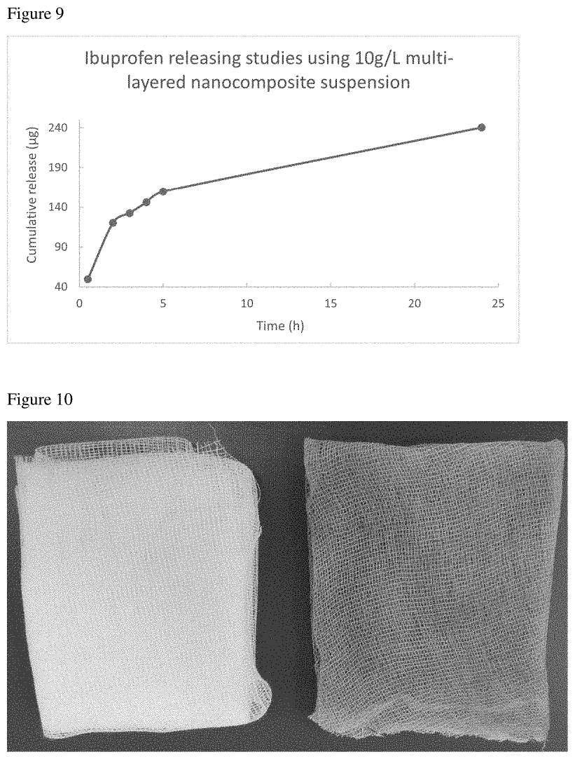

Release of ibuprofen from polymer-nanoparticle composite was done in pH 7.4 phosphate buffer medium. 10 mL of multifuctional nanocomposite of concentration 10 g/L was dialyzed against phosphate buffer (100 mL). UV-Vis spectrum of the buffer was analyzed at different time intervals to determine the drug release kinetics. The drug release profile obtained at 37° C. temperature is shown in FIG. 9.

Bactericidal efficacy of the multi-layered Ibuprofen-loaded polymer nanoparticle composites loaded with Tetracaine was tested against a spectrum of Gram (+Ve) and Gram (−Ve) bacteria. For comparison, bactericidal efficacy was compared with a composition of only the nanoparticle material (NED: CuO₍₁₋ₓ₎ZnOₓ). As shown in the following table, the bactericidal properties of the nanocomposite-polymer composition were either unchanged with addition of the chitosan and alginate polymer layers or improved, with respect to efficacy against E. coli.

| | Bactericidal effect (% Killing), 100 ppm concentration | | | |
|---|---|---|---|---|
| | Escherichia | Klebsiella | Staphylococcus | Listeria |
| Sample | coli | pneumoniae | aureus | monocytogenes |
| NED | 86.58 | 99.96 | 99.9 | 99.99 |
| ChiAlgChiAlgNED | 96.52 | 99.97 | 99.999 | 99.90 |

### Example 8: Preparation of Multi-Functional, Multiple Drug-Releasing, Polymer-Nanoparticle Composite with Antibacterial and Hemostasis Promoting Properties

This example describes preparation of an embodiment of the described nanocomposite composition which contains two anesthetic drugs Tetracaine and Ibuprofen in Na-Alginate and chitosan layers respectively. The multifunctional nanocomposite presented here is a representative example to demonstrate antibacterial, hemostatic properties and ability to load and release multiple drugs.

Step 1, coating the nanoparticle with a Tetracaine-loaded Na-Alginate polymer layer was performed as in previous examples.

Step 2, coating the nanoparticle-polymer-drug composite with Ibuprofen-loaded chitosan polymer layer was performed as in previous examples.

The result of Steps 1 and 2 is a nanoparticle having a two polymer layers: a Tetracaine-loaded Na-Alginate polymer layer that in turn is coated with an Ibuprofen-loaded chitosan polymer layer.

The drug loading efficiency of the polymer nanoparticle composite was approximately 50% for tetracaine and 14% for ibuprofen.

### Example 9: Embedding First Aid Gauze with Multi-Functional Polymer-Nanoparticle Composite

This example is another illustration of embedding the described polymer-nanoparticle compositions in gauze substrate for use in first aid and other wound care applications.

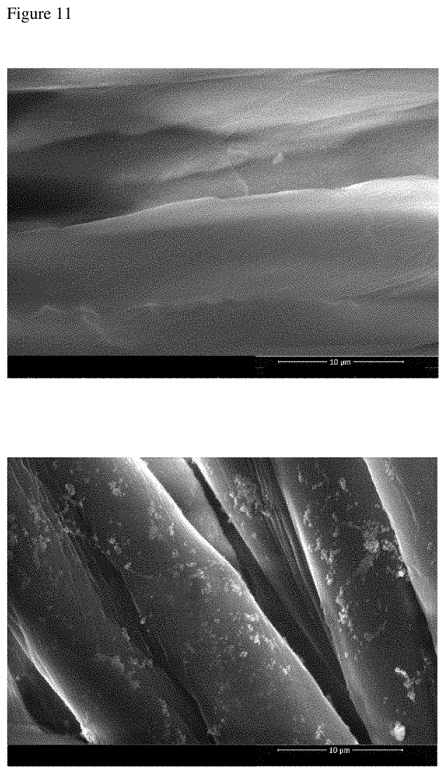

A multi-functional polymer-nanoparticle composite having a net positive charge on its outer layer from among those such as described in the previous examples, was embedded in gauze suitable for first aid applications, by using roll-to-roll machinery. The gauze is moved between the rolls at 10 to 100 meters per minute. Between the rolls, a rotational spray machine spray coats the gauze with multi-functional polymer nanoparticle composite at the rate of 2 to 30 mL/m2. Concentration of the multi-functional polymer nanoparticle composite suspension is varied between 0.1 to 2 g/L to obtain embedding percentage by weight in the range of 0.1 to 1.0 wt %. Multi-functional polymer-nanoparticle composite embedded gauze was dried using blow drier, and collected as a roll. FIG. 10 shows photographic images of coated (0.5 g/L) and uncoated gauze samples. FIG. 11 shows scanning electron microscopy (SEM) images of uncoated (top panel) and coated (bottom panel) fibers.

To evaluate the bactericidal efficacy of the nanocomposite in the first aid gauze against Escherichia coli, the standard AATCC 100 testing protocol was performed (see Askew, Peter D. Chimica oggi 27, no. 1 (2009): 16-20). Coated gauze showed more than >99.999% reduction of Escherichia coli within 2 hours exposure at 36° C.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for treatment of a wound in a subject comprising,
administering to the subject a composition comprising:
an antimicrobial metal oxide nanoparticle;
a first polymer layer coating the antibacterial metal oxide nanoparticle;
an external polymer layer coating the polymer-coated nanoparticle; and
optionally at least one additional polymer layer between the first polymer layer and the external polymer layer,
wherein the first polymer layer is optionally a hemostasis-promoting polymer and/or comprises a pharmaceutical agent; and
wherein the external polymer layer and the optional at least one additional polymer layer is a hemostasis-promoting, mucoadhesive polymer, and/or comprises a pharmaceutical agent that is the same or different from the pharmaceutical agent of the first polymer layer.

2. The method of claim 1, wherein the antibacterial metal oxide nanoparticle core comprises CuO, ZnO, Ag₂O, TiO₂, MgO, or Fe₂O₃.

3. The method of claim 1, wherein the metal oxide nanoparticle core comprises a doped metal oxide.

4. The method of claim 3, wherein the doped metal oxide comprises Zn doped CuO, Cu doped ZnO, Ag doped TiO₂, or Mg doped ZnO.

5. The method of claim 1, wherein the metal oxide nanoparticle inhibits bacteria, fungi, and/or viruses.

6. The method of claim 1, wherein the first, external, and/or additional polymers are hemostasis-promoting.

7. The method of claim 6, wherein each of the hemostasis-promoting polymers is independently calcium alginate, polylysine, oxidized cellulose, chitosan and modifications thereof, gelatin, or thiomers thereof.

8. The method of claim 1, wherein the pharmaceutical agent is selected from a pain reliever, local anesthetic and/or non-steroidal anti-inflammatory drug (NSAID).

9. The method of claim 1, wherein the external polymer layer has a positive or a negative zeta potential.

10. The method of claim 1, wherein the composition is administered to the subject in or on a wound dressing.

11. The method of claim 1, wherein the composition is formulated for topical administration.

12. A wound treatment composition comprising:
an antimicrobial metal oxide nanoparticle;
a first polymer layer coating the antibacterial metal oxide nanoparticle;
an external polymer layer coating the polymer-coated nanoparticle; and
optionally at least one additional polymer layer between the first polymer layer and the external polymer layer,
wherein the first polymer layer is optionally a hemostasis-promoting polymer and/or comprises a pharmaceutical agent; and
wherein the external polymer layer and the optional at least one additional polymer layer is a hemostasis-promoting, mucoadhesive polymer, and/or comprises a pharmaceutical agent that is the same or different from the pharmaceutical agent of the first polymer layer.

13. The composition of claim 12, wherein the antibacterial metal oxide nanoparticle core comprises CuO, ZnO, Ag₂O, TiO₂, MgO, or Fe₂O₃.

14. The composition of claim 12, wherein the metal oxide nanoparticle core comprises a doped metal oxide.

15. The composition of claim 14, wherein the doped metal oxide comprises Zn doped CuO, Cu doped ZnO, Ag doped TiO₂, or Mg doped ZnO.

16. The composition of claim 12, wherein the first, external, and/or additional polymers are hemostasis-promoting.

17. The composition of claim 16, wherein each of the hemostasis-promoting polymers is independently calcium alginate, polylysine, oxidized cellulose, chitosan and modifications thereof, gelatin, or thiomers thereof.

18. The composition of claim 12, wherein the pharmaceutical agent is selected from a pain reliever, local anesthetic and/or non-steroidal anti-inflammatory drug (NSAID).

19. A wound dressing comprising the composition of claim 12.

20. A topical formulation comprising t the composition of claim 12.
